(19) United States
(12) Patent Application Publication
(10) Pub. No.: US 2011/0039767 A1
(43) Pub. Date: Feb. 17, 2011

(54) FOOD COMPOSITIONS

(76) Inventors: Arie Gijsbert Nieuwenhuizen, Utrecht (NL); Klaas Roelof Westerterp, Maastricht (NL); Margaretha Adeleida Bernadette Veldhorst, Maastricht (NL); Ananda Hochstenbach-Waelen, Landgraaf (NL); Robert-Jan Brummer, Orebro (SE); Margriet-Sjoukje Westerterp-Plantenga, Maastricht (NL); David Jason Mela, Schiedam (SE)

Correspondence Address: NIXON & VANDERHYE, PC, 901 NORTH GLEBE ROAD, 11TH FLOOR, ARLINGTON, VA, 22203, US

(21) Appl. No.: 12/667,752

(22) PCT Filed: Jul. 3, 2008
(86) PCT No.: PCT/NL2008/050449

§ 371 Date: Sep. 29, 2010

(30) Foreign Application Priority Data
Jul. 6, 2007 (EP) 07111941.6

(51) Int. Cl. A61K 38/39; C07K 14/78; A61P 3/04

(52) U.S. Cl. 514/4.9; 530/356

(57) ABSTRACT

The present inventors have found that collagen hydrolysate can be favorably used for the preparation of an edible composition for limiting voluntary food intake and hence are suitable for prevention and treatment of overweight and obesity.

### FIELD OF THE INVENTION

The present invention relates to food composition that upon ingestion increase feelings of satiety or reduce feelings of hunger and thus limit voluntary food intake. The food composition are thus suitable for treating and/or preventing overweight and/or obesity.

### BACKGROUND OF THE INVENTION

The prevalence of obesity has increased worldwide to epidemic proportions. When viewing the Dutch population over the period 1993-1997, 40.2% of the men and 25.0% of the women aged from 37-43 yrs. were regarded to suffer from overweight (25<Body Mass Index/BMI (kg/m²)<29.9). Moreover, in the same study, 8.5% of the men and 9.3% of the women were regarded obese (BMI>30 kg/m²) [1].

Since overweight and obesity are associated with an increased risk for a number of serious diseases, such as coronary heart diseases, hypertension, non-insulin dependent diabetes pulmonary dysfunction, and certain types of cancer, the development of strategies for weight loss is important, because even a modest weight loss (5-10% of initial body weight) markedly reduces the risk for mortality and morbidity [2].

Strategies for weight loss are based on accomplishing a negative energy balance, either by increasing energy expenditure or by decreasing energy intake. In this respect, high protein diets have gained considerable interest. Of all macronutrients, proteins have shown to be the most satiating [3]. In addition, high protein diets improve fat oxidation and body composition during weight loss as well as during weight regain [4, 5].

Still, not much is known about the role of protein quality in its satiating effects. Only a limited number of small human studies describe a comparison of different protein sources with respect to their effects on satiety or food intake, with mixed results. One of the most well-known satiety cases have been made for alpha-lactalbumin [6]. This tryptophan-rich protein has been suggested to be highly satiating, because of its putative potency to increase the levels of brain serotonin (which is an endogenous mediator of satiety). It is noted that plasma tryptophan is a precursor for serotonin.

Tryptophan-rich proteins have been claimed for preventing or treatment of overweight and/or obesity, cf. US 2006/0257497.

WO 2005/023017 describes a food composition comprising collagen hydrolysate and a tryptophan source. According to this document, the consumption of the composition may occur as a part of a dietary plan, such as to reduce or control body weight.

The tryptophan-containing food compositions for reducing or controlling body weight according to the state of the art are associated with a number of disadvantages.

Firstly, many natural sources of tryptophan originate from dairy proteins, such as casein or whey protein. Such natural sources of tryptophan typically contain lactose. It is known that a significant fraction of the world population is intolerant to lactose. Thus, there exists a need for lactose-free food compositions for reducing or controlling body weight.

Secondly, the ingestion of a natural source of tryptophan, and/or of L-tryptophan, will normally not only influence satiety or appetite. An increased availability of plasma tryptophan may, through its effect on brain serotonin levels, also affect a person's state of mind. Besides influencing appetite, serotonin is believed to also play an important role in the regulation of anger, aggression, body temperature, mood, sleep, vomiting, and sexuality. Therefore, current edible compositions, rich in natural sources of tryptophan and/or L-tryptophan, which are marketed for reducing and/or controlling body weight may in effect have other physiological effects which may not be desired at all times.

Thirdly, many natural sources of tryptophan have a certain characteristic taste. WO2005/023017 mentions such natural tryptophan sources as whey concentrate, yeast extract, tomato powder, and whole egg powder. The formulator of edible compositions comprising a natural tryptophan source is faced with the daunting challenge to carefully select the natural tryptophan source which is suitable for the type of food product to be consumed without negatively interfering with the taste which is expected and desired by the consumer.

### SUMMARY OF THE INVENTION

Surprisingly, it has been found that collagen hydrolysate, which is essentially free from tryptophan, can be used for the preparation of an edible composition for limiting voluntary food intake. In addition, it has been found that collagen hydrolysate can be used for the preparation of an edible composition for reducing a feeling of hunger. Said edible composition is substantially free from a natural source of tryptophan. In a further preferred embodiment, said composition is further essentially free from L-tryptophan. The invention also relates to an edible composition comprising collagen hydrolysate for use in a method for treatment and/or prevention of obesity, wherein the edible composition is essentially free from a natural tryptophan source.

The present invention also provides an edible composition comprising collagen hydrolysate, preferably in an amount of from 0.1-75 wt. % (based on the total weight of the composition), said composition being essentially free from a natural tryptophan source.

The edible compositions and/or the uses thereof according to the present invention advantageously allow for
1. the formulation of satiety-enhancing edible compositions without using possibly allergenic substances, which compositions can thus be consumed by a larger fraction of the population, and/or
2. the ingestion of edible compositions having the desired effects of limiting appetite and/or reducing a feeling of hunger without significantly affecting those physiological properties which are related to enhanced serotonin levels, and/or
3. the formulation of essentially bland-tasting satiety-enhancing edible compositions, from which a wide array of food products can be derived of which the desired flavor profile can be easily determined using natural and/or artificial aroma components, herbs, spices, etc. known to the person skilled in the art, and/or
4. controlling overweight and/or obesity, in particular. preventing overweight and/or obesity and/or treating overweight and/or obesity.

It has hitherto not been possible to provide satiety-enhancing foodstuffs having the specific advantages of the present invention as outlined above.

### DETAILED DESCRIPTION OF THE INVENTION

### Definition of Collagen Hydrolysate

The food compositions of the invention comprise collagen hydrolysate. Collagen hydrolysate is defined as hydrolyzed gelatin, which is obtained by controlled hydrolysis of gelatin obtained from animal collagen. The terms ‘collagen hydrolysate’ and ‘hydrolyzed gelatin’ are used interchangeably throughout this description. The hydrolyzed gelatin is preferably obtained by enzymatic or chemical hydrolysis of gelatin. Preferably, the hydrolyzed gelatin is obtained from Type A gelatin. Commercially available sources of hydrolyzed gelatin may also be referred to as hydrolysated gelatin, non-gelatinizing gelatin or gelatin hydrolysate.

The hydrolyzed gelatin according to the invention preferably has an average molecular weight between 1 and 20 kDalton. Even more preferably, the average molecular weight is between 2 and 10 kDalton. A peptide with a chain length of approximately 25-40 amino acids on average is preferred. Thus it is preferred that of the collagen hydrolysate that is used at least 75% wt. %, preferably at least 80 wt. %, more preferably at least 90 wt. %, even more preferably at least 95 wt. % has a length of between 25-40 amino acids.

It is within these boundaries that the current invention can be applied, advantageously using high levels of the hydrolyzed gelatin with minimal effects on viscosity and/or flavor of the edible composition. Preferably, the hydrolyzed gelatin is used for preparing liquid edible compositions, such as drinks and shakes. In another preferred embodiment, hydrolyzed gelatin is used to prepare solid or semi-solid compositions, such as nutritional bars.

Depending on factors such as the desired mouthfeel, viscosity, and/or gel strength, the hydrolyzed gelatin may be advantageously employed together with a non-hydrolyzed gelatin to prepare the edible compositions according to the present invention. The non-hydrolyzed gelatin may comprise any type of food-grade non-hydrolyzed gelatin, such as the gelatin which is generally employed for the preparation of gelled foodstuffs, such as gums, liquorice, marshmallows, meringues, chewy fruit candies, fillings, toffees, puddings etc. The molecular weight of said non-hydrolyzed gelatin preferably resides between 20 and 200 kDalton. The gel strength of 6% solutions of the gelatin preferably corresponds to between 50 and 325 bloom.

### Use, Technical Effects, Unit Dose, Effective Amount

As is further illustrated in the Examples, the present inventors have found that after consumption by a subject of an edible composition comprising collagen hydrolysate, said subject voluntarily limited intake of a “common food” which was offered at a later stage for ad libitum consumption. The edible composition is essentially free from a natural tryptophan source. The “common food” is a food product known to and regularly consumed by the subject. The “common food” could generally be any food that could be part of a food routine of a person and may comprise for instance rice, bread, eggs, meat, fish, vegetables or pasta, and which is preferably not a food product for controlling body weight.

In addition, the present inventors have found that said subject experiences a reduced feeling of hunger in the period between consumption of the edible consumption according to the invention and the intake of the common food.

Thus, the present inventors have found that collagen hydrolysate can be used in the preparation of an edible composition for limiting voluntary food intake, for reducing a feeling of hunger, and for the treatment and/or prevention of overweight and for the treatment and/or prevention of obesity.

Preferably, the edible composition is administered in such a dosing regime to provide between 5 and 200 grams of collagen hydrolysate for consumption by a subject per day, this is called the daily dose. Preferably, the edible consumption is administered such that a subject consumes between 10 and 100 grams of collagen hydrolysate per day. Alternatively or simultaneously, the edible composition according the present invention preferably provides for 5-50%, even more preferably for 10-50%, of the advised total calories intake per day. Herein, the advised total calories intake per day is the estimated energy requirement as referred to in the Dietary Guidelines for Americans 2005, published by the U.S. Department of Health and Human Services and the U.S. Department of Agriculture, cf. www.healthierus.gov/dietaryguidelines. Although the advised total calories intake depends on gender, age and physical activity level of the subject, roughly speaking, the advised total calories intake per day is 2000 kcal for a grown-up female and 2500 kcal for a grown-up male. For practical purposes, in three different preferred embodiments, the advised total calories intake per day may hence be understood as 2000 kcal, 2500 and 2250 kcal, the latter value preferably relating to an edible composition which could be marketed as a one-suits-all type of product for controlling bodyweight.

As it turns out, a minimum amount of collagen hydrolysate should be consumed in order to have a noticeable effect on limiting voluntary food intake and/or on reducing a feeling of hunger. If too much collagen hydrolysate is consumed per day, especially if the edible composition according to the invention is essentially free from a natural source of tryptophan and/or L-tryptophan, the subject may refuse the consumption of other protein sources comprising tryptophan, so that he may become deprived with this essential amino acid. Alternatively, the subject would need to ingest tryptophan sources (including protein sources) in such an amount that his daily protein intake would be at risk to become unbalanced. In this respect, it should be noted that the addition of L-tryptophan to foodstuffs for human consumption is not freely allowed in most countries, so that tryptophan is to be commonly ingested as a natural tryptophan source, which is usually (almost by definition) proteinaceous. Therefore, in one embodiment, the edible composition is essentially free from L-tryptophan.

Conveniently, the amount of the edible composition that is to be ingested by, or is administered to, a subject is defined as a unit dose. Herein, a “unit dose” refers to a portion which is intended to be consumed in a single sitting, for example preferably in a single portion. Thus preferably a unit dose comprises the daily dose of collagen hydrolysate, however in one embodiment a daily dose may be divided in two or more unit doses, together comprising the daily dose of collagen hydrolysate, which are intended to be ingested in two or more sittings.

It is preferred that the edible composition is administered once to three times a day. In other words, the edible composition is preferably administered to a subject in the form of one to three unit doses which are administered and consumed within a time span of 24 h. The one to three unit doses together preferably provide between 5 and 200 grams, more preferably between 10 and 100 grams of collagen hydrolysate per day.

In a further preferred embodiment, the edible composition is administered once a day, preferably as a breakfast or as a lunch. In this embodiment, the edible composition is provided as one unit dose which preferably provide between 5 and 200 grams, more preferably between 10 and 100 grams of collagen hydrolysate. It is further preferred that in this embodiment, the edible composition provides between 1 and 50%, preferably between 1 and 25%, of the advised total calories intake per day. In this embodiment, the subject receives the collagen hydrolysate in a sufficient amount to reduce voluntary food intake during the next meal. Thus, if the unit dose is provided as a breakfast, the subject will voluntarily reduce his/her food intake during lunch, and if the unit dose is provided as a lunch, the subject will voluntarily reduce his/her food intake during dinner. The food which is consumed during the meal following the intake of the unit dose may be part of a usual, preferably healthy and balanced, food routine of the subject, so that preferably, over a period of 24 hours, the subject will voluntarily reduce its food intake whilst receiving a balanced diet containing the essential amino acids, despite the fact that over the day, one meal (which is provided as one unit dose of the edible composition according to the present invention) will be preferably deprived of tryptophan. According to this embodiment, obesity and/or overweight of the subject can be treated and/or prevented by providing the composition according to the present invention, which limits the voluntary food intake of the subject and preferably reduces a feeling of hunger, whilst a healthy and balanced diet may be provided which provides all essential nutrients and amino acids which are needed on a daily basis.

It is further preferred that the unit dose is consumed within the period of time which is usually spent by people to consume a similar type of “common food” which is part of their own food routine, and which is preferably not a food product for controlling body weight. For example, and preferably, the duration of a single sitting ranges between 10 seconds and 5 minutes for the consumption of an edible composition in the form of a drink, between 30 seconds and 10 minutes for the consumption of an edible composition in the form of a nutritional bar, between 1 minute and 30 minutes for the consumption of an edible composition in the form of a meal replacement, etcetera.

The size of a unit dose will depend upon the type of composition. For beverages and soups, the typical size of a unit dose (or serving size) is in the range of from 100 to 500 ml. For puddings, the typical serving size is in the range of from 75 g to 300 g. For bars the typical serving size is in the range of from 20 g to 70 g.

A unit dose preferably provides for 25-400 kcal.

### Amount of Collagen Hydrolysate in the Edible Composition

Depending on the desired size of the unit dose and the consistency, mouthfeel and type of the edible composition, the edible composition according to the present invention comprises between 0.1-75 wt. % of collagen hydrolysate, based on the total weight of the composition. Preferably, the collagen hydrolysate provides at least 2-50% of the total calories of the composition.

Preferably, the amount of collagen hydrolysate in the edible composition is from 1 to 25 wt. %, even more preferably, from 2-10 wt. %, based on the total weight of the composition. It appears that the amount of collagen hydrolysate according to the indicated ranges allows for the formulation of edible compositions which are ready to eat and/or ready to drink, and which are acceptable to the consumer, in particular in terms of mouthfeel and/or flavor of the composition and/or size of the unit dose, and which provide sufficient amounts of collagen hydrolysate per unit dose which is consumed by a subject to have a noticeable effect on limiting voluntary food intake and/or reducing a feeling of hunger and hence for the treatment and/or prevention of overweight and/or obesity.

### Natural Tryptophan Source

As defined herein, a natural tryptophan source is any protein-containing food component which provides for more than 50 mg of tryptophan per 100 g of the food component. In particular a natural tryptophan source is any protein containing more than 50 mg of tryptophan per 100 g of the said protein. In the context of the present invention, L-tryptophan is not a natural source of tryptophan.

Collagen hydrolysate is not a natural source of tryptophan. Furthermore, gelatin is not a natural source of tryptophan. In the context of this invention a collagen hydrolysate preferably contains less than 50 mg tryptophan per 100 g of collagen hydrolysate, in particular it contains preferably less than 45, more preferably less than 40, more preferably less than 35, more preferably less than 30, more preferably less than 25, more preferably less than 20, more preferably less than 15, more preferably less than 10, more preferably less than 5 mg of tryptophan per 100 g of collagen hydrolysate.

Examples of natural tryptophan sources include dairy and vegetable proteins. Sources which include higher than average tryptophan levels include whey protein, egg white (egg albumin), whole egg powder, milk protein, yeast extract, tomato powder, brazil nut protein, inca peanut protein, soybean protein, cottonseed protein and sunflower protein.

### Macronutrient Composition

Preferably, the edible composition according to the present invention comprises:
a. collagen hydrolysate, in such an amount that it provides at least 2-50% of the total calories of the composition, and
b. optionally, another protein source, which is not a tryptophan source, and
c. a fat source, in such an amount that it provides between 0.1 and 50% of the total calories of the composition, and
d. a carbohydrate source, in such an amount that it provides between 0.1 and 85%, more preferably between 0.1 and 80%, of the total calories of the composition.

In one embodiment, the edible composition is further essentially free from a natural tryptophan source. In another embodiment, the composition is further essentially free of L-tryptophan.

A particularly preferred edible composition comprises
i. collagen hydrolysate, in such an amount that it provides between 5 and 35%, even more preferably between 10 and 25% of the total calories of the composition, and
ii. a fat source, in such an amount that it provides between 5 and 50%, even more preferably between 20 and 35% of the total calories of the composition, and
iii. a carbohydrate source, in such an amount that it provides between 10 and 75%, even more preferably between 40 and 60%, for example about 55%, of the total calories of the composition.

These preferred ranges in particular allow the food formulator to compose a suitable unit dose of an edible composition having a balanced macronutrient profile which may provide for sufficient collagen hydrolysate to promote limiting voluntary food intake and/or reducing a feeling of hunger and that are suitable for the treatment and/or prevention of overweight and/or obesity.

### Carbohydrate

The carbohydrates are preferably present in an amount of from 2 to 60% by weight based on the weight of the composition, more preferably from 5 to 40 wt. %. The amount of carbohydrate in the food composition will vary according to the composition and also, where required, according to national or regional legislation.

Any suitable carbohydrates may be included in the food compositions. Suitable examples include starches such as are contained in rice flour, flour, tapioca flour, tapioca starch and whole wheat flour, modified starches or mixtures thereof. If a sweet taste is desired, generally, the food compositions will be naturally sweetened and this is preferred as a source of carbohydrate. Suitable natural sweeteners include sugars and sugar sources such as sucrose, lactose, glucose, fructose, maltose, galactose, corn syrup (including high fructose corn syrup), sugar alcohols, maltodextrins, high maltose corn syrup, starch, glycerin, brown sugar and mixtures thereof.

Levels of sugars and sugar sources preferably result in sugar solids levels of up to 40 wt %, preferably from 5 to 20 wt % based on the weight of the food compositions. The artificial sweeteners mentioned below as optional ingredients may also be used the whole, or a part, of the carbohydrate source.

The compositions preferably contain a total amount of from 0.1 to 20% wt. % of dietary fiber, more preferably 0.2 to 15 wt. %, most preferably 0.5 to 10 wt. %, especially 1 to 7 wt. %

These amounts include any biopolymer thickening agent present in the composition that is a dietary fiber. Suitable fiber sources which may be included in the food compositions of the invention, in addition to the biopolymer thickening agent, include fructo-oligosaccharides such as inulin, soy fiber, fruit fiber (e.g. apple fiber, oat fiber), celluloses and mixtures thereof.

The food compositions may optionally comprise one or more polysaccharides. Preferably, these optional other polysaccharides are used for thickening purposes and/or for achieving nutritional benefits.

Preferably, these optional other polysaccharides are selected from ionic, preferably anionic, non-starch polysaccharides and neutral non-starch polysaccharides. In one embodiment, the food compositions comprise resistant starch.

Preferred ionic non-starch polysaccharides are alginates having an L-guluronic acid content of less than 60% of the total uronic acid units in the alginate, pectins including amidated pectins, carrageenans, xanthans, gellans, furcellarans, karaya gum, rhamsan, welan, gumghatti, gum arabic and salts or mixtures thereof. Suitable salts include the alkaline and alkaline earth metal salts, especially sodium, potassium, calcium or magnesium salts.

The food composition may optionally additionally comprise a neutral non-starch polysaccharide. Especially preferred neutral non-starch polysaccharides are galactomannan, guar gum, locust bean gum, tara gum, ispaghula, beta-glucans, konjacglucomannan, methylcellulose, gum tragacanth, detarium, tamarind or mixtures thereof. Of these, galactomannan, guar gum, locust bean gum and tara gum are especially preferred.

### Fat

The compositions of the invention preferably comprise food-grade fats, preferably in an amount of up to 30% by weight based on the weight of the composition, more preferably from 0.1 to 20 wt. %, most preferably from 0.2 to 10% wt. %, especially from 0.5 to 5 wt. %

The amount of fat will vary according to the composition and also, where required, according to national or regional legislation. For example, EC Directive 96/8/EC states that for meal replacement products the energy derived from fat shall not exceed 30% of the total energy of the product. Additionally, the linoleic acid (in the form of glycerides) shall not be less than 1 g.

Any food fat may be used for example, animal fats including fish oils, vegetable fats including plant oils, nut oils, seed oils, or mixtures thereof. Monounsaturated and/or polyunsaturated fats and mixtures thereof are especially preferred although saturated fats can be used for taste reasons, e.g. butter, although these are less preferred on health grounds. Preferred polyunsaturated fats include omega 3 fatty acids, especially docosahexaenoic acid (DHA, C20: 5) and/or eicosapentaenoic acid (EPA, C22: 5).

Preferred omega 3 fatty acids include the following C18: 3, C18: 4, C20: 4, C20: 5, C22: 5 and C22: 6.

Preferably the fat is selected from vegetable fats, such as for example, cocoa butter, illipe, shea, palm, palm kernel, sal, soybean, safflower, cottonseed, coconut, rapeseed, canola, corn and sunflower oils, tri and di-glyceride oils including linoleic acids and conjugated linoleic acids, linolenic acids, and mixtures thereof.

### Optional Ingredients

The edible compositions of the invention may comprise one or more of the following optional ingredients.

The compositions of the invention may further comprise encapsulated satiety agents which are predominantly released in the intestines. Suitable satiety agents include lipids, especially mono-, di- or tri-glycerides, their free fatty acids, their food salts, their non-glyceryl esters, hydrolyzable in the presence of gastro-intestinal enzymes, and mixtures thereof. These satiety agents may be encapsulated in any suitable cross-linked encapsulating agent whereby they are predominantly released in the intestines.

Encapsulant materials comprising gelatin and at least one of gum arabic, carrageenan, agar agar, alginate or pectins, especially gelatin and gum arabic, have been found to be very suitable. These encapsulated satiety agents may be included in suitable amounts.

The composition may comprise one or more emulsifiers. Any suitable emulsifier may be used, for example lecithins, egg yolk, egg-derived emulsifiers, diacetyl tartaric esters of mono, di or tri glycerides or mono, di, or triglycerides. The composition may comprise of from 0.05 to 10% by weight, preferably from 0.5% to 5% wt of the emulsifier based on the weight of the product.

If it is desired to include a bulking agent in the nutrition bars, within or external to the protein nuggets, a preferred bulking agent is inert polydextrose. Other conventional bulking agents which may be used alone or in combination therewith include maltodextrin, sugar alcohols, corn syrup solids, sugars or starches. Total bulking agent levels in the protein nuggets, and in the nutritional bars of the invention, will preferably be from about 0% to 20 wt %, preferably 5% to 16%. Polydextrose may be obtained under the brand name Litesse.

Flavorings are preferably added to the food compositions in amounts that will impart a mild, pleasant flavor. The flavoring may be any of the commercial flavors typically employed. When a non-savory taste is desired the flavors are typically selected from varying types of cocoa, pure vanilla or artificial flavor, such as vanillin, ethyl vanillin, chocolate, malt, mint, yogurt powder, extracts, spices, such as cinnamon, nutmeg and ginger, mixtures thereof, and the like. It will be appreciated that many flavor variations may be obtained by combinations of the basic flavors. When a savory taste is desired the flavors are typically selected from varying types of herbs and spices. Suitable flavorings may also include seasoning, such as salt, and imitation fruit or chocolate flavors either singly or in any suitable combination.

Flavorings which mask off-tastes from vitamins and/or minerals and other ingredients are preferably included in the food compositions. Other flavorings such as fruit flavorings may also be used, with an example being pineapple flavoring.

Among fiber sources which may be included in the food compositions of the invention are fructo-oligosaccharides such as inulin, soy fiber, fruit fiber, guar gum, gum arabic, gum acacia, oat fiber, cellulose and mixtures thereof. Preferably, fiber sources are present in the product at greater than 0.5 wt % and do not exceed 20 wt %, especially do not exceed 15 wt % or 10 wt %. As indicated above, additional bulking agents such as maltodextrin, sugar alcohols, corn syrup solids, sugars, starches and mixtures thereof may also be used. Total bulking agent levels in the products of the invention, including fibers and other bulking agents, will preferably be from about 0% to 80%, especially from 10 to 80 wt %, most preferably from 15-70 wt. %

The food compositions may comprise one or more conventional colourants, in conventional amounts as desired.

The composition may also comprise 0.1 to 5% by weight of food buffering salts based on the weight of the composition. Any suitable food buffering salt may be used.

The composition may comprise one or more cholesterol lowering agents in conventional amounts. Any suitable, known, cholesterol lowering agent may be used, for example isoflavones, phytosterols, soy bean extracts, fish oil extracts, tea leaf extracts.

The composition may optionally comprise, in suitable amounts, one or more agents which may beneficially influence (post-prandial) energy metabolism and substrate utilization, for example caffeine, flavonoids (including tea catechins, capsaicinoids and carnitine).

The composition may comprise up to 10 or 20% by weight, based on the weight of the composition, of minor ingredients selected from added vitamins, added minerals, herbs, spices, antioxidants, preservatives or mixtures thereof. Preferably the compositions comprise of from 0.05 to 15% by weight, more preferably 0.5 to 10% of these ingredients.

The composition preferably comprises added vitamins selected from at least one of; Vitamin A Palmitate, Thiamine Mononitrate (Vitamin B1), Riboflavin (Vitamin B2), Niacinamide (Vitamin B3), d-Calcium Pantothenate (Vitamin B5), Vitamin B6, Vitamin B11, Cyanocobalamin (Vitamin B12), biotin, Ascorbic acid (Vitamin C), Vitamin D, Tocopheryl Acetate (Vitamin E), Biotin (Vitamin H), and Vitamin K. The composition also preferably comprises added minerals selected from at least one of calcium, magnesium, potassium, zinc, iron, cobalt, nickel, copper, iodine, manganese, molybdenum, phosphorus, selenium and chromium. The vitamins and/or minerals may be added by the use of vitamin premixes, mineral premixes and mixtures thereof or alternatively they may be added individually. The vitamins and minerals must be provided in the composition in a format which allows them to be absorbed by the consumer and must hence have good bioavailability.

In particular the food compositions preferably comprise alkaline metals such as sodium and/or potassium.

Calcium is preferably present in the food compositions in amounts of from 5 to 150% of the amounts given in the European Commission Directive 96/8/EC of 26 Feb. 1996 on foods intended for use in energy-restricted diets for weight reduction, more preferably about 10 to 135% per unit dose. Any suitable calcium source may be used.

It is preferred that the food compositions comprise potassium, especially in an amount of at least 300 mg of potassium per unit dose of the food composition, more preferably 400-1000 mg, most preferably 500-700 mg. Any suitable potassium source may be used.

One or more of the above-mentioned vitamins and minerals are preferably present at amounts of from 5 to 45% of the amounts given in the above European Commission Directive 96/8/EC, especially 5 to 40%, most especially 10 to 30%.

Generally the nutrition bars of the invention will be naturally sweetened. Natural sources of sweetness include sucrose (liquid or solids), glucose, fructose, and corn syrup (liquid or solids), including high fructose corn syrup and high maltose corn syrup and mixtures thereof. Other sweeteners include lactose, maltose, glycerin, brown sugar and galactose and mixtures thereof. Levels of sugars and sugar sources preferably result in sugar solids levels of up to 50 wt %, preferably from 5 to 18 wt %, especially from 10 to 17 wt % of the nutrition bar.

Any of the artificial sweeteners well known in the art may be used, such as aspartame, saccharine, Alitame (obtainable from Pfizer), acesulfam K (obtainable from Hoechst), cyclamates, neotame, sucralose, mixtures thereof and the like. The sweeteners are used in varying amounts.

### Type of Composition

The edible, in particular food composition according to the present invention may be of any type, for example a liquid or spoonable composition, a bar product or a cereal-type product such as an extruded pasta- or rice-type product. In one embodiment the composition may also be in a substantially dehydrated form, to which dehydrated composition the consumer must add a liquid, preferably water, in order to prepare a food product which is ready to eat or ready to drink.

Compositions comprising high protein levels, e.g. from 25-75 wt %, are particularly suitable for preparing solid compositions, in particular nutritional bars (for direct consumption), and substantially dehydrated compositions, including reconstitutable powders. The dehydrated compositions, including reconstitutable powders, may also essentially consist of collagen hydrolysate, accompanied with instructions to reconstitute the powder with a liquid, preferably water, or a fluid food product for example a fluid or low viscous (diet) dairy product for weight management purposes. Nutritional bars having a collagen hydrolysate content between 25 and 75 wt % are especially preferred for animal feed.

Especially preferred food compositions are those which are intended to be used as part of a weight loss or weight control plan, such as a meal replacer product.

Suitable types of liquid or spoonable compositions according to the invention include drinks, oil-in-water emulsions (such as dressings), creams, desserts such as mousses, custards, puddings, non-dairy yogurts; frozen confectionery including ice cream, water ices, sorbets, and non-dairy frozen yoghurts; breakfast type products; shakes, soups, sauces, sport drinks, etc.

Frozen confectionery is considered to be a spoonable food composition because even though it is in a frozen state, it still meets the definition of a spoonable composition herein at the temperature at which it is consumed.

Preferably the amount of water in the liquid or spoonable compositions (including any water present in other ingredients) is in the range of from 20 to 95% wt, more preferably from 30 to 90 wt %.

Alternatively, the food composition may be a nutritional bar or a cereal-type product such as an extruded pasta- or rice-type product.

According to an aspect of the invention, the food composition may be dehydrated.

When a composition is described as being dehydrated, this means that the total water content in the composition is less than 10 wt. %. For improved shelf stability, a water content of less than 6 wt. % is preferred. Such dehydrated compositions may conveniently be in the form of reconstitutable powders, with a liquid being added and the mixture stirred in order to generate a food product which is ready to drink or ready to eat. For example, powder can be mixed with hot or cold water in order to make soups or shakes Dehydrated compositions according to the invention may also be pasta-type meals, to which liquid is again added to generate the final product.

The amount of liquid relative to the amount of food composition will vary depending on the food product desired. Exemplary amounts are from 1 to 99 wt % food composition and 1 to 99 wt % liquid, preferably 1 to 50 wt % food composition and 50 to 99 wt % liquid. The food product is preferably a pasta-type product, a soup or a shake.

The terms “meal replacer(s)” or “meal replacement product(s)” as used herein also include compositions which are eaten as part of a meal replacement weight loss or weight control plan, for example snack products which are not intended to replace a whole meal by themselves but which may be used with other such products to replace a meal or which are otherwise intended to be used in the plan; these latter products typically have a calorie content in the range of from 25-400, preferably of from 50-200 kilocalories per unit dose.

Meal replacers are generally used by consumers following a calorie controlled diet and are especially preferred food compositions according to the invention. They have been found to be especially suitable as they can provide good satiety effects combined with restricted calorie content in a convenient form.

### Manufacture

The composition of the invention may be prepared by any suitable conventional technique according to the type of food composition. Such techniques are well known to those skilled in the art and do not need to be described further here but may include mixing, blending, extrusion homogenizing, high-pressure homogenizing, emulsifying, dispersing, or extruding. The compositions may be subject to a heat treatment step, for example pasteurization or U.H.T. treatment.

### EXAMPLES

### Example 1

### Comparative Demonstration of the Effects of Collagen Hydrolysate on Satiety and Voluntary Food Intake

### Objective

The objective of the present study was to evaluate the effect of casein, soy protein, whey protein with glycomacropeptide (whey-1), whey protein without glycomacropeptide (whey-2), alpha-lactalbumin, collagen hydrolysate, or collagen hydrolysate with added tryptophan (with Trp added to the level present in the alpha-lactalbumin) in either a normal or a high protein breakfast on EI during lunch, which was offered three hours after breakfast, and on possible related satiety measures collected following breakfast.

### Subjects

Thirty healthy male and female volunteers (Body Mass Index 22-35 kg/m2, age 18-45 year) were recruited by advertisements in local newspapers and on notice boards at the University of Maastricht (The Netherlands). They underwent a screening including medical history, measurement of body weight and height and cognitive restrained eating using a Dutch translation of the Three Factor Eating Questionnaire (TFEQ, [7]). Twenty-four subjects were selected on being in good health, non-smokers, non-vegetarian, not cognitively dietary restraint, not using medication apart from oral contraceptives and at most moderate alcohol users. A written informed consent was obtained from these participants and the study protocol was approved by the Medical Ethical Committee of the Academic Hospital Maastricht.

### Study Design

A randomized, single-blind, within-subject experimental study was performed. All subjects came to the University on 14 occasions, separated by at least three days. On each test day subjects received a subject-specific standardized breakfast. Three hours after breakfast an ad libitum lunch was offered; appetite ratings were obtained until six hours after breakfast.

### Breakfast

### Protein Sources; Abbreviated Terms

Breakfast was offered as a custard, with either casein, soy, whey protein with glycomacropeptide (whey-1), whey protein without glycomacropeptide (whey-2), alpha-lactalbumin, collagen hydrolysate, or collagen hydrolysate+Trp. Herein, “collagen hydrolysate+Trp” refers to collagen hydrolysate with added tryptophan, the tryptophan being added to the level present in the alpha-lactalbumin.

Hereinafter, “alpha-lactalbumin” may be abbreviated as “alpha-lac”; “collagen hydrolysate” may be abbreviated as “col” and “collagen hydrolysate+Trp” may be abbreviated as “col+Trp”.

### Custards

These proteins were present in the custard as a single protein source. This breakfast contained each of these 7 single protein sources in random order, and in two different macronutrient compositions. The macronutrient composition was either protein/carbohydrate/fat (C/P/F): 10/55/35 en % (normal protein diet) or protein/carbohydrate/fat: 25/55/20 en % (high protein diet). Herein, en % (provided by each macronutrient) is short for “energy percent” (provided by each macronutrient), and refers to the percentage of the total calories of the composition which is provided by each macronutrient.

The 14 different types of custards had tapioca starch as carbohydrate source and sunflower oil as fat source and were lemon-vanilla flavored. The color, taste, and viscosity did not differ significantly among the 14 types of custards; the breakfasts differed only in protein composition and were produced by NIZO Food Research bv. (Ede, The Netherlands). Formulatory details regarding the custards which were provided for breakfast are represented in Table 1. Amino acid analyses of the protein sources which were used to prepare the custards according to Table 1 are reported in Table 2.

The breakfast contained 20% of daily dietary energy requirements (DDER), calculated as basal metabolic rate (BMR), according to the equations of Harris-Benedict, multiplied by an activity index of 1.75. Thus, the BMR (kCal/day) was calculated according to the following equations:

Male: BMR=66,473+5,003H+13,752W−6,755A 1.

Female: BMR=655,096+1,850H+9,563W−4,676A 2.

H (height) in cm, W (weight) in kg, A (age) in year.
Nota bene: the DDER should not be confused with the advised total calories intake (ATCI) per day. The DDER is used within the framework of the study to provide statistically significant results which are quantitatively comparable amongst subjects having a different BMR. The DDER has nothing to do with recommendations for establishing a healthy energy balance.

### Lunch

Lunch consisted of Turkish bread (400 g) with egg salad (400 g) with 13/41/46 En % protein/carbohydrate/fat. The Turkish bread was offered as finger food (i.e. in small pieces) in order to further promote that the food intake of the subjects indeed took place on an “ad libitum” basis.

### Study Protocol

After an overnight fast from 22.00 h, the protocol started at 08.30 h with scoring appetite ratings. Breakfast was offered (t=0 minutes) and completed within 20 minutes. With the first and the last bite taste perception was scored. Appetite ratings were completed at 30,160 60, 90, 120, and 180 minutes after breakfast. Immediately after completing the questionnaire at 180 minutes, subjects were offered an ad libitum lunch and were instructed to eat just as much till they were satiated. With the first and the last bite of the lunch taste perception was scored. Appetite ratings then were completed at 210, 240, 300, and 360 minutes after breakfast. Subjects were allowed to drink maximally three glasses of water spread over the entire test period and were allowed to go home four hours after breakfast; the last two moments of rating were completed at home and returned on the next visit.

### Measurements

### Energy Intake (EI)

Lunch was weighed before and after eating and EI was calculated by multiplying the difference of the weight of the lunch by the energy value of the lunch as determined by the product labels.

### Appetite Profile

To determine the appetite profile, hunger, fullness, satiety, and desire to eat were rated on 100 mm Visual Analogue Scales (mm VAS), anchored with ‘not at all’ and ‘extremely’. Subjects were instructed to rate the appetite dimensions by marking the scale at the point that was most appropriate to their feeling at that time.

### Taste Perception

Taste perception profiles of the custards and lunch were assessed after the first and the last bite using 100 mm Visual Analogue Scales (VAS), anchored with ‘not at all’ and ‘extremely’ on the aspects: pleasantness, sweetness, sourness, saltiness, bitterness, savouriness, crispiness, and creaminess.

### Statistical Analysis

Data are presented as mean changes from baseline+standard error to the mean (SEM), unless otherwise indicated. The area under the curve (AUC) of changes from baseline till 180 minutes (AUC180) or 360 minutes (AUC360) was calculated using the trapezoidal method. A repeated measures ANOVA was carried out to determine possible differences between 25 and 10% energy from protein within the same type of protein and possible differences between the different types of protein within the 25% and 10% energy from protein conditions. Bonferonni correction was used for multiple comparisons. Regression analysis was performed to determine the relationships between the difference in EI between two different breakfasts and the difference in AUC of hunger or satiety after these two different breakfasts. Glucose, insulin, GLP-1, and ghrelin concentrations between different protein types within one concentration were compared using the Mann-Whitney U test (Veldhorst M A B, Nieuwenhuizen A G, Hochstenbach-Waelen A, et al. Effects of casein-, soy-, or whey with or without GMP-protein breakfasts in two concentrations on amino acid, satiety, and ‘satiety’ hormone responses; submitted). A p-value <0.05 was regarded as statistically significant. Statistical procedures were performed using StatView 5.0 (SAS 200 Institute Inc., USA, 1998).

### Results

### Subject Characteristics

Mean age of the subjects (10 male, 14 female) was 25±2 year, and their body weight was 72.8±2.2 kg (BMI: 24.8±0.5 kg/m²). The TFEQ scores were 5.9±0.6 (F1, cognitive restraint), 4.7±0.5 (F2, disinhibition), and 4.2±0.6 (F3, hunger). The mean energy content of the breakfast (20% of calculated daily Total Energy Expenditure) was 2.39±0.06 MJ.

### Taste Perception Breakfast

Pleasantness of taste of the custards with the first bite was sufficient with a mean value of 55±5 mm without statistically significant differences between custards. Sensory specific satiety after eating the breakfast expressed as delta pleasantness of taste was on average −12±5 mm; again there were no statistically significant differences between custards.

### FIGURES

FIG. 1 shows energy intake (kJ) at lunch after consumption of a custard, wherein the a source of protein is provided by casein, soy, whey-1, whey-2, alpha-lactalbumin, collagen hydrolysate, or col+TRP, and wherein the single source of protein provides for 10 En % (A) or 25 En % (B) of the custard. The compositions of the custards are represented in Table 1. Values are represented as means ±SEM for the results obtained for 24 subjects (men and women).

ANOVA repeated measures with Bonferonni correction are shown; “a” is significantly different from “b” (p<0.05), “a1” is significantly different from “b1” (p<0.05), “a2” is significantly different from “b2” (p<0.05)

FIG. 2 shows changes in satiety and hunger (mmVAS) after consumption of a custard, wherein a single source of protein is provided by casein, soy, whey-1, whey-2, alpha-lactalbumin, collagen hydrolysate, or col+TRP, and wherein the single source of protein provides for 10 En % (A) or 25 En % (B) of the custard. The compositions of the custards are represented in Table 1. Values are represented as means ±SEM for the results obtained for 24 subjects (men and women).

ANOVA repeated measures with Bonferonni correction are shown, *p<0.05.

Legend to FIG. 2. ---Δ casein 10%, ---∘ soy 10%, ---□ whey-1 10%, ---⋄ whey-2 10%, ---x alpha-lactalbumin 10%, ---▪ collagen hydrolysate 10%, --- col+Trp 10%; —Δ casein 25%, —∘ soy 25%, —□ whey-1 25%, —⋄ whey-2 25%, —x alpha-lactalbumin 25%, —▪ collagen hydrolysate 25%, — col+Trp 25%

FIG. 3 shows the relation of difference in appetite ratings (satiety or hunger, mmVAS·h) and difference in EI between two custards given for breakfast to 24 subjects (men and women), the custards containing as a single source of protein casein, soy, whey-1, whey-2, alpha-lactalbumin, collagen hydrolysate, and col+Trp, at both 10 En % or 25 En % from protein. The compositions of the custards are represented in Table 1. Values are represented as means obtained for 24 subjects (men and women). Legend to FIG. 3. Difference in appetite ratings and EI between a breakfast with: col+Trp 10%—soy 10% (r=−0.470, p<0.05), ▴collagen hydrolysate 10%—whey-2 10% (r=−0.641, p<0.001), ♦ col+TRP 10%—whey-2 10% (r=−0.446, p<0.05), ▪ col+Trp 25%—soy 25% (r=−0.571, p<0.01), ∘ alpha-lactalbumin 10%—whey-1 10% (r=0.531, p<0.01), Δ collagen hydrolysate 10%—whey-1 10% (r=0.481, p<0.05), ⋄ col+TRP 10%—whey-2 10% (r=0.414, p<0.05), □ col+TRP 25%—soy 25% (r=0.458, p<0.05)

### Energy Intake

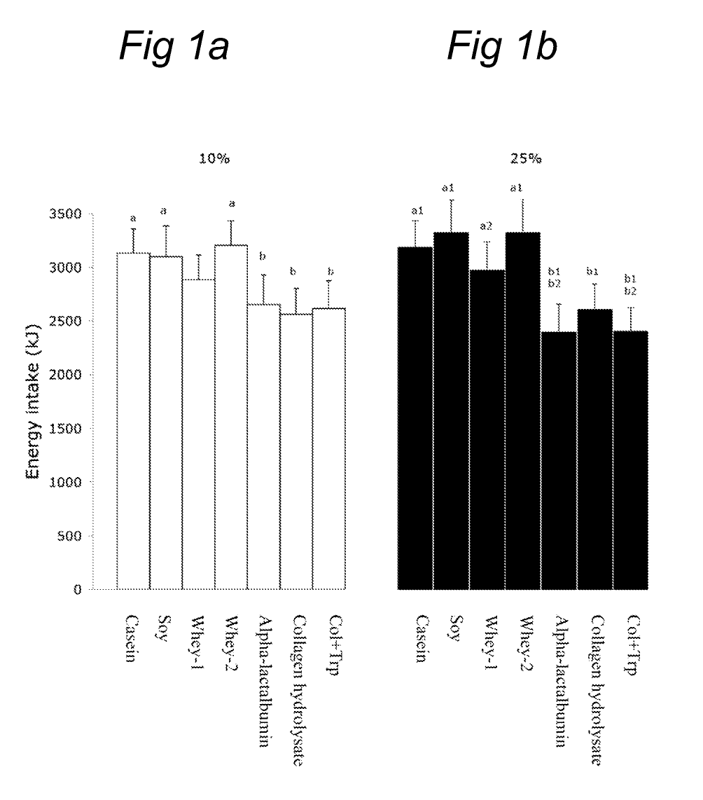

There were no differences in ad libitum energy intake (EI) at lunch between a breakfast with 25% of energy from protein compared with a breakfast with 10% of energy from the same protein type. After a breakfast with 10% of energy from protein, ad libitum EI at lunch was 0.54 MJ (17%) lower after a breakfast with alpha-lactalbumin, collagen hydrolysate, or collagen hydrolysate+Trp compared with a breakfast with casein, soy, or whey-2 (p<0.05, p<0.05, p<0.01, p<0.01, p<0.01, p<0.01, p<0.01, p<0.05, and p<0.01 resp., FIG. 1). After a breakfast with 25% of energy from protein, ad libitum EI at lunch was 0.78 MJ (24%) lower after a breakfast with alpha-lactalbumin, collagen hydrolysate, or collagen hydrolysate+Trp compared with a breakfast with casein, soy, or whey-2 (p<0.05, p<0.01, p<0.01, p<0.01, p<0.001, p<0.001, p<0.01, p<0.01, and p<0.001 resp., FIG. 1). EI at lunch was also 0.55 MJ (19%) lower after a breakfast with alpha-lactalbumin or collagen hydrolysate+Trp compared with a breakfast with whey-1 (p<0.01 and p<0.01 resp., FIG. 1).

### Satiety and Hunger

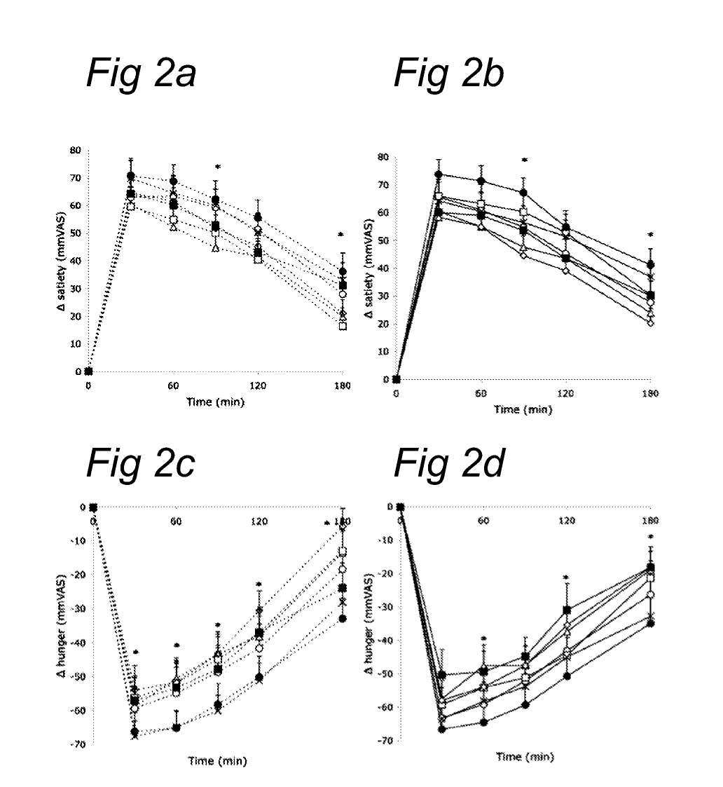

There were various significant differences in the change in satiety or hunger between the seven different breakfasts at the level of 10 or 25% of energy from protein, these are presented in FIG. 2.

### AUC180

The AUC over the first three hours after breakfast, i.e. the AUC180 of satiety was increased after a breakfast with 10% of energy from alpha-lactalbumin compared with one with casein or whey-1 (Table 3). The AUC180 of satiety was increased after a breakfast with collagen hydrolysate+Trp compared with one with casein or whey-1. The AUC180 of hunger was more decreased after a breakfast with alpha-lactalbumin compared with one with casein, whey-1, whey-2, or collagen hydrolysate, and was also more decreased after a breakfast with collagen hydrolysate+Trp compared with one with casein, whey-1, whey-2, or collagen hydrolysate. The AUC180 of satiety was increased after a breakfast with 25% of energy from whey-1 compared to one with whey-2 and after a breakfast with collagen hydrolysate+Trp compared with one with casein, soy, whey-2, or collagen hydrolysate. The AUC180 of hunger was more decreased after a breakfast with soy or alpha-lactalbumin compared with one with collagen hydrolysate, and was more decreased after a breakfast with collagen hydrolysate+Trp compared with one with casein, whey-2, or collagen hydro lysate (all differences p<0.05). The order of magnitude of differences in AUC180 satiety or hunger was 1700-2500 mmVAS·h (Table 3).

### Correlations

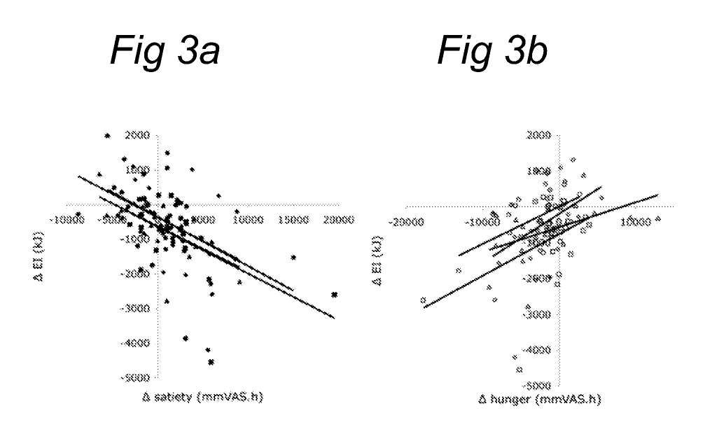

Comparison of the different protein breakfast types at a concentration of 10% of energy from protein revealed that the difference in EI at lunch between a breakfast with collagen hydrolysate+Trp and a breakfast with soy a function was of the difference in the AUC180 of satiety between those two breakfasts (r=−0.470, p<0.05, FIG. 3), the difference in EI at lunch between a breakfast with collagen hydrolysate and a breakfast with whey-2 was a function of the differences in the AUC180 of satiety or the AUC180 of hunger between those two breakfasts (r=−0.641, p<0.001; and r=0.481, p<0.05 resp., FIG. 3), and the difference in EI at lunch between a breakfast with collagen hydrolysate+Trp and whey-2 was a function of the differences in the AUC180 of satiety or the AUC180 of hunger between those two breakfasts (r=−0.446, p<0.05; r=0.414, p<0.05 resp., FIG. 3).

Comparison of the different protein types at a concentration of 25% of energy from protein revealed that the difference in EI at lunch between a breakfast with collagen hydrolysate+Trp and a breakfast with soy a function was of the difference in the AUC180 of satiety or the AUC180 of hunger between those two breakfasts (r=−0.571, p<0.01; r=0.458, p<0.05 resp., FIG. 3).

### Blood Parameters

The comparison of glucose concentrations, obtained during the previous study (Veldhorst M A B, Nieuwenhuizen A G, Hochstenbach-Waelen A, et al. Effects of casein-, soy-, or whey with or without GMP-protein breakfasts in two concentrations on amino acid, satiety, and ‘satiety’ hormone responses; submitted), revealed that the AUC of the glucose response was increased after a breakfast with 10% of energy from collagen hydrolysate (138±13 mmol/l·h) compared with a breakfast with 10% of energy from whey-1 (99±14 mmol/l·h, p<0.05). The AUC of the insulin response was increased after a breakfast with 10% of energy from alpha-lactalbumin (6683±711 mU/l·h), collagen hydrolysate (7391±723 mU/l·h), or collagen hydrolysate+Trp (6744±711 mU/l·h) compared with a breakfast with soy (4936±468 mU/l·h, p<0.05, p<0.05, and p<0.001 respectively). The AUC of the insulin response was also increased after a breakfast with 25% of energy from alpha-lactalbumin (9080±988 mU/l·h), collagen hydrolysate (7698±847 mU/l·h), or collagen hydrolysate+Trp (8227±1033 mU/l·h) compared with a breakfast with casein (4792±980 mU/l·h, p<0.001, p<0.05, and p<0.01, respectively) and after a breakfast with 25% of energy from alpha-lactalbumin (9080±988 mU/l·h) compared with a breakfast with 25% of energy from soy (7520±929 mU/l·h, p<0.05). The AUC of the GLP-1 response was increased after a breakfast with 25% of energy from collagen hydrolysate+Trp (462±105 μmol/l·h) compared with a breakfast with 25% of energy from casein (161±90 μmol/l·h, p<0.05) or soy (195+72 μmol/l·h, p<0.05). There were no differences in ghrelin responses between the different protein types.

### Discussion

Ad libitum EI at lunch was reduced after a breakfast with 10% of energy from alpha-lactalbumin, collagen hydrolysate, or collagen hydrolysate+Trp compared with a breakfast with 10% of energy from casein, soy, or whey-2. After a breakfast with 25% of energy from protein, ad libitum EI at lunch was reduced after a breakfast with alpha-lactalbumin, collagen hydrolysate, or collagen hydrolysate+Trp compared with a breakfast with casein, soy, or whey-2 and also after a breakfast with alpha-lactalbumin or collagen hydrolysate+Trp compared with a breakfast with whey-1. EI was substantially decreased with ˜0.7 MJ; a reduction of ˜20%. The iso-energetic custards were of the same color and viscosity and did not differ in taste, so differences are only due to the type of protein. To explain the differences in EI we explored differences in appetite ratings, and ‘satiety’ hormones. Satiety at three hours after breakfast, just before the ad libitum lunch, was significantly increased after a breakfast with either 10% or 25% of energy from alpha-lactalbumin, collagen hydrolysate, and/or collagen hydrolysate+Trp compared with casein, soy, whey-1, and/or whey-2. The inverse pattern was observed for hunger. Differences in appetite ratings between two treatments were correlated to the difference in EI between those two treatments, so reduced EI indeed was related straightforwardly to increased satiety. Alpha-lactalbumin, collagen hydrolysate, and collagen hydrolysate+Trp thus were more satiating three hours after breakfast than casein, soy, whey-1, and whey-2 and this resulted in a decreased ad libitum EI at lunch.

A mechanism for the increased satiety and decreased EI may be the increased insulin response after a breakfast with alpha-lactalbumin, collagen hydrolysate, or collagen hydrolysate+Trp compared with a breakfast with casein or soy. Insulin is a metabolic satiety signal that may explain the increased perceived satiety. There was also an increased GLP-1 response after a breakfast with 25% of energy from collagen hydrolysate+Trp compared with a breakfast with 25% of energy from casein or soy. Previously, GLP-1 has been found to inhibit appetite and reduce food intake in normal-weight men. GLP-1 possibly exerts its effects via a combination of inhibition of gastric emptying and activation of brain GLP-1 receptors that limits food intake. The increased GLP-1 response after a breakfast with 25% of energy from collagen hydrolysate+Trp compared with casein or soy may contribute to an increased satiety response and reduced food intake.

Our results show that with breakfasts with different protein types a significant difference in EI at lunch is likely to be achieved if the difference in induced satiety is considerable. The differences in satiety between the protein types consumed at breakfast that induced a different EI at lunch were 15 to 25 mm on a Visual Analogue Scale; a ˜40% increased satiety. When differences in appetite ratings were smaller, but still statistically significant, no difference in EI was observed. Apparently differences in appetite ratings of less than 30% are not large enough to induce significant differences in EI at a later occasion. To obtain a significant reduction in EI satiety needs to be increased with at least 15 to 25 mmVAS or 40%. Moreover, a significant reduction of EI apparently has to be consistently and sufficiently large. Here we observed the difference of ˜0.7 MJ or ˜20%.

So, in addition to the required differences in magnitude of satiety, a considerable difference in EI needs to be present. Since we observed this phenomenon with various comparisons between different proteins, the magnitude effect may well be more generally applicable. One of the objectives of this study was to compare EI at lunch after a high and a normal protein breakfast from the same protein type; however no significant differences in EI were observed between breakfasts with 25 and 10 En % from the same type of protein. Nevertheless there were some significant differences in satiety and/or hunger between a breakfast with casein, soy, or whey-2 with 25 or 10% of energy from protein. These differences had a magnitude of 10 to 15 mm VAS; too small to induce a reduction in EI at a lunch three hours after the breakfast.

Timing of the moment when an ad libitum meal is offered is important in evaluating the satiating properties of protein. The most sensitive time point to offer lunch after providing subjects with the custards according to the present study appeared to be three hours after breakfast. Significant differences in satiety were present and resulted in a 20% reduction of EI after an alpha-lactalbumin, collagen hydrolysate, or collagen hydrolysate+Trp custard.

The results of the calculation of the AUC till 360 minutes after breakfast revealed remarkable results in that the AUC360 is similar to the AUC till 180 minutes after breakfast. So even when the subjects ate less during lunch, satiety still was increased and hunger was decreased after a breakfast with alpha-lactalbumin, collagen hydrolysate, or collagen hydrolysate+Trp.

Summarizing, alpha-lactalbumin, collagen hydrolysate, or collagen hydrolysate+Trp containing breakfasts caused a 20% reduced EI at lunch compared to either a casein, soy, or whey-2 breakfast, both at the level of 10% and 25% of energy from protein.

Thus, the group of proteins selected from alpha-lactalbumin, collagen hydrolysate, and collagen hydrolysate+Trp is 30-50% more satiating than other proteins (casein, soy, whey-1, and whey-2) and induces a related 17-24% reduction of subsequent energy intake.

### CONCLUSIONS

The results can be summarized as follows:
Within the group of seven protein sources, collagen hydrolysate and alpha-lactalbumin, when administered to a group of subjects as part of a custard breakfast providing 20% of the daily dietary energy requirement, reduced voluntary food intake and/or reduced a feeling of hunger to an above-average extent. When L-tryptophan was added to the collagen hydrolysate in levels corresponding to those present in the compositions comprising alpha-lactalbumin, voluntary food intake was not further reduced as compared with the composition comprising collagen hydrolysate without added L-tryptophan. Above effects occurred both in a normal (C/P/F: 35/10/55 en %) and in a high protein (C/P/F: 35/25/40 en %) custard meal.
Without being bound to theory, the underlying mechanism for satiety enhancement by collagen hydrolysate is apparently independent of induction of satiety as mediated by a serotonin receptor. It is further noted that the timing of the peak in plasma amino acid levels after consumption of both collagen hydrolysate and alpha-lactalbumin was not earlier than after consumption of whey, or later than after consumption of casein. Hence, the results cannot solely be explained by differences in digestion and/or absorption rates. Moreover, the viscosity profiles of all different custards used in the experiments were designed to match as closely as possible. For example, the viscosity at 50 s⁻¹, as determined at room temperature, is in the order of 10³ Pa·s for each custard. Therefore, effects on satiety due to viscosity differences can be excluded.
Thus, it is concluded that surprisingly, collagen hydrolysate, when provided as protein source in a meal both under normal and high protein conditions, has significantly higher appetite- and/or voluntary-food-intake-reducing effects when compared to the regular dietary protein sources whey protein, casein and soy protein. The effect on limiting voluntary food intake associated with the consumption of the edible composition comprising collagen hydrolysate, which composition is essentially free of a natural source of tryptophan, is within statistical error the same as the effect of the edible composition comprising alpha-lactalbumin, which composition provides a natural source of tryptophan.

Thus, collagen hydrolysate can be used for the preparation of an edible composition for limiting voluntary food intake and/or for reducing a feeling of hunger, said composition being essentially free from a natural tryptophan source.

### Example 2

### Preparation of a Bar

A candy bar for one unit dose can be prepared using the following ingredients:

| | |
|---|---|
| Carbohydrate (sucrose) | 5 g |
| Carbohydrate (starch) | 10 g |
| Vegetable fat | 4 g |
| Protein (collagen hydrolysate) | 12 g |
| Fibres | 9 g |

The collagen hydrolysate is obtained as Solugel LMC/3, PB Gelatins GmbH, Germany.

### Example 3

### Preparation of a Rehydratable Powder

A rehydratable powder for one unit dose can be prepared using the following ingredients:

| | |
|---|---|
| Carbohydrate (sugars) | 21 g |
| Carbohydrate (maltodextrin) | 8 g |
| Fat (saturated) | 1 g |
| Fat (mono-unsaturated) | 3 g |
| Fat (poly-unsaturated) | 4 g |
| Protein (collagen hydrolysate) | 10 g |
| Fibres | 8 g |

The collagen hydrolysate is obtained as Solugel LMC/3, PB Gelatins GmbH, Germany.

### Example 4

### Another Comparative Test

In a single-blind, randomised cross-over design the effects of gelatin on subjective hunger perceptions were compared with a standard dietary protein (casein).

Subjects (n=22) reported, after an overnight fast, to the laboratory on 2 occasions, separated by at least one week. On each occasion, at t=0 (8:00 a.m.), 270 and 630 min, they received a breakfast, lunch and diner, containing 20%, 40% and 40% of their mean daily energy need, respectively. All meals had a normal macronutrient distribution, and contained 10% protein, 55% carbohydrate and 35% fat. The protein sources in all meals differed on both occasions, and were either casein (control) or gelatin.

At frequent time points during the day, subjective feelings of hunger were assessed using visual analogue scales (not shown), expressed as hunger feeling (mm VAS) as a function of time (h). After each meal, hunger scores dropped for both protein sources. The drop in hunger scores was more pronounced when gelatin was the protein source, compared with the control. In addition, when the meal contained gelatin as protein source, hunger scores returned to pre-meal levels later then when casein was the protein source. Also, at any point in time, hunger scores were significantly lower when gelatin was the protein source, as compared with the control.

To further demonstrate the satiety-enhancing effect of the gelatin, the area under the curve (AUC) for the hunger scores (mm VAS) from t=0 to 13.5 hours was determined, showing the cumulative hunger feelings over time. This AUC was determined as approx. −300 (mm VAS)·h for the control, whereas the AUC was approx. −450 (mm VAS)·h for the meal having gelatin has the protein source. This result clearly indicates that the subjects perceived less feelings of hunger throughout the day when they consumed gelatin as a protein source, when compared with a standard dietary protein, i.e., casein.

TABLE 1
Formulatory details of the edible compositions used with the test panel

| | normal protein | high protein content |
|---|---|---|
| | content (10 En %) | (25 En %) |
| | Amount weighed in | Amount weighed in |
| Raw material | (grams) | (grams) |
| Custard based on whey-1 | | |
| Volactive Ultra Whey 90 | 409.2 | 1029.2 |
| (from cheese whey) | | |
| Starch (VA50T) | 651 | 596.75 |
| Starch (Perfectamyl 3108) | 127.1 | 173.6 |
| Carrageenan | 13.95 | 13.95 |
| Sucrose | 1224.5 | 1224.5 |
| Pre-emulsion*¹ | 2813.25 | 1883.25 |
| Citrus flavor | 2.325 | 2.325 |
| Vanilla flavor | 15.5 | 15.5 |
| Water | 10243.175 | 10560.925 |
| TOTAL | 15500 | 15500 |
| Custard based on whey-2 | | |
| WPC 80 (from acid whey) | 392.32 | 988.8 |
| Starch (VA50T) | 806.4 | 806.4 |
| Starch (Perfectamyl 3108) | 0 | 0 |
| Carrageenan | 14.4 | 14.4 |
| Sucrose | 1264 | 1264 |
| Pre-emulsion*¹ | 2960 | 1992 |
| Citrus flavor | 2.4 | 2.4 |
| Vanilla flavor | 16 | 16 |
| Water | 10544.48 | 10916 |
| TOTAL | 16000 | 16000 |
| Custard based on casein | | |
| Ca-caseinate | 382.85 | 933 |
| Starch (VA50T) | 527 | 187.5 |
| Starch (Perfectamyl 3108) | 241.8 | 523.5 |
| Carrageenan | 13.95 | 13.5 |
| Sucrose | 1224.5 | 1170 |
| Pre-emulsion*¹ | 2821 | 1770 |
| Citrus flavor | 2.325 | 2.25 |
| Vanilla flavor | 15.5 | 15 |
| Water | 10271.075 | 10385.25 |
| TOTAL | 15500 | 15000 |
| Custard based on soy | | |
| Soy protein Supro 590 | 399.125 | 1038.4 |
| Starch (VA50T) | 310 | 0 |
| Starch (Perfectamyl 3108) | 426.25 | 730.4 |
| Carrageenan | 13.95 | 14.4 |
| Sucrose | 1224.5 | 1264 |
| Pre-emulsion*¹ | 2836.5 | 1940.8 |
| Citrus flavor | 4.65 | 4.8 |
| Vanilla flavor | 46.5 | 48 |
| Water | 10238.525 | 10959.2 |
| TOTAL | 15500 | 16000 |
| Custard based on | | |
| Alpha-lactalbumin | | |
| Alpha-lactalbumin | 429.25 | 1209 |
| Starch (VA50T) | 856.8 | 912 |
| Starch (Perfectamyl 3108) | 0 | 47.5 |
| Carrageenan | 15.3 | 17.1 |
| Sucrose | 1343 | 1501 |
| Pre-emulsion*¹ | 3111 | 2394 |
| Citrus flavor | 2.55 | 2.9 |
| Vanilla flavor | 17 | 19 |
| Water | 11225.1 | 12897.6 |
| TOTAL | 17000 | 19000 |
| Custard based on | | |
| collagen hydrolysate and | | |
| col + Trp*² | | |
| Collagen hydrolysate (Solugel | 520.8 | 1373.68 |
| LMC/3, PB Gelatins GmbH) | | |
| Starch (VA50T) | 1080 | 1120 |
| Starch (Perfectamyl 3108) | 0 | 0 |
| Carrageenan | 21 | 30.8 |
| Sucrose | 1659 | 1738 |
| Pre-emulsion*¹ | 3906 | 2816 |
| Citrus flavor | 6.3 | 6.6 |
| Vanilla flavor | 42 | 44 |
| Water | 13761.3 | 14855.72 |
| TOTAL | 21000 | 22000 |

*¹The pre-emulsion is prepared from 8.5 kg sunflower oil, 170 g of a lactic ester emulsifier, and 34 kg of water;
*²An amount of 20 g of tryptophan has been added to 21,000 g of the custard based on collagen hydrolysate to obtain the custard based on col + Trp.

TABLE 2
Amino acid analyses of the protein sources which were used
to prepare the edible compositions according to Table 1.

| | whey-1 | whey-2 | casein | soy | alpha-lac | col |
|---|---|---|---|---|---|---|
| | Composition (g of amino acid per | | | | | |
| Amino Acids | 100 g of protein source) | | | | | |
| Cystine | 2.42 | 3.14 | 0.37 | 1.06 | 4.92 | 0.03 |
| Methionine | 2.11 | 2.28 | 2.73 | 1.10 | 1.18 | 0.85 |
| Aspartic acid | 10.1 | 11.2 | 6.4 | 9.8 | 15.4 | 5.6 |
| (=asn + asp) | | | | | | |
| Hydroxyproline | — | — | — | — | — | 12.0 |
| Threonine | 6.6 | 4.71 | 3.85 | 3.23 | 4.88 | 1.86 |
| Serine | 4.35 | 3.93 | 5.1 | 4.34 | 4.08 | 3.26 |
| Glutamic acid | 16.7 | 16.8 | 20.3 | 16.1 | 13.5 | 10.1 |
| (glx = gln + glu) | | | | | | |
| Proline | 5.6 | 4.33 | 9.8 | 4.26 | 2.43 | 13.9 |
| Glycine | 1.53 | 1.68 | 1.69 | 3.49 | 2.53 | 24.6 |
| Alanine | 4.65 | 4.65 | 2.71 | 3.59 | 2.39 | 9.3 |
| Valine | 5.4 | 5.0 | 6.0 | 4.19 | 4.42 | 2.26 |
| Isoleucine | 6.2 | 5.6 | 4.77 | 4.38 | 5.8 | 1.52 |
| Leucine | 9.9 | 12.3 | 8.7 | 7.1 | 11.0 | 2.95 |
| Tyrosine | 2.68 | 3.50 | 5.1 | 3.37 | 4.26 | 0.47 |
| Phenylalanine | 2.72 | 3.45 | 4.66 | 4.62 | 4.02 | 1.87 |
| y-amino butyric | — | — | — | — | 0.32 | — |
| acid | | | | | | |
| Histidine | 1.69 | 2.10 | 2.74 | 2.34 | 2.77 | 0.91 |
| Ornithine | — | — | — | — | — | 0.32 |
| Lysine | 8.8 | 10.2 | 7.3 | 5.4 | 10.5 | 3.85 |
| Arginine | 2.42 | 2.99 | 3.93 | 6.8 | 1.82 | 8.4 |
| Tryptophan | 1.73 | 2.24 | 1.16 | 1.12 | 3.84 | n.d. |

n.d. = could not be detected

TABLE 3
Satiety and hunger ratings after consumption of a custard, expressed as
AUC180 and AUC360 (mmVAS.h). As a single source of protein of the custard,
casein, soy, whey-1, whey-2, alpha-lactalbumin, collagen hydrolysate, and col + TRP are
provided in both 10 En % and 25 En %. The compositions of the custards are
represented in Table 1.

| | Satiety | | Hunger | |
|---|---|---|---|---|
| | AUC180 | AUC360 | AUC180 | AUC360 |
| Casein 10 En % | 7190 ± 986^{ab} | 15350 ± 1890^{ab} | −6699 ± 910^{ab} | −13940 ± 1869^{ab} |
| Casein 25 En % | 7513 ± 1096^{e} | 15596 ± 2134^{f} | −6821 ± 882ⁱ | −14652 ± 1713^{g} |
| Soy 10 En % | 8723 ± 1005 | 18097 ± 1928 | −7307 ± 1092 | −15425 ± 2155 |
| Soy 25 En % | 8307 ± 1163^{f} | 17418 ± 2136 | −7966 ± 992^{j} | −16964 ± 1949^{h} |
| Whey-1 10 En % | 7250 ± 1038^{cd} | 15542 ± 2062^{cd} | −6645 ± 978^{cd} | −14433 ± 2015^{cd} |
| Whey-1 25 En % | 8972 ± 931^{g} | 18658 ± 1962^{g} | −7574 ± 820 | −15702 ± 1773 |
| Whey-2 10 En % | 8006 ± 836 | 16686 ± 1747^{e} | −6006 ± 945^{ef} | −13259 ± 1939^{ef} |
| Whey-2 25 En % | 7165 ± 981^{gh} | 14998 ± 1948^{gh} | −6918 ± 935^{k} | −14588 ± 1854ⁱ |
| Alpha-lac 10 En % | 9094 ± 969^{ac} | 18831 ± 1845^{ac} | −8922 ± 746^{aceg} | −18635 ± 1552^{ace} |
| Alpha-lac 25 En % | 8853 ± 973 | 17894 ± 1967 | −8274 ± 861^{l} | −16871 ± 1847^{j} |
| Collagen hydrolysate | 8198 ± 956 | 17657 ± 1878 | −7121 ± 1146^{gh} | −15349 ± 2301 |
| 10 En % | | | | |
| Collagen hydrolysate | 8046 ± 983ⁱ | 16466 ± 2015ⁱ | −6273 ± 1026^{jlm} | −13017 ± 2126^{hjk} |
| 25 En % | | | | |
| Col + Trp 10 En % | 9648 ± 1002^{bd} | 19948 ± 1961^{bde} | −8926 ± 911^{bdfh} | −18478 ± 1900^{bdf} |
| Col + Trp 25 En % | 100075 ± 849^{efhi} | 20652 ± 1817^{fhi} | −9046 ± 878^{ikm} | −18565 ± 1898^{gik} |

Values are represented as means ±SEM for the results obtained for 24 subjects (men and women). ANOVA repeated measures with Bonferonni correction: the same letter within a column indicates a significant difference between two treatments (p<0.05)

### REFERENCES

1. Visscher, T. L., D. Kromhout and J. C. Seidell, Long-term and recent time trends in the prevalence of obesity among Dutch men and women. Int J Obes Relat Metab Disord, 2002. 26(9): p. 1218-24.
2. Goldstein, D. J., Beneficial health effects of modest weight loss. Int J Obes Relat Metab Disord, 1992. 16(6): p. 397-415.
3. Stubbs, R. J. and S. Whybrow, Energy density, diet composition and palatability: influences on overall food energy intake in humans. Physiol Behav, 2004. 81(5): p. 755-64.
4. Westerterp-Plantenga, M. S., The significance of protein in food intake and body weight regulation. Curr Opin Clin Nutr Metab Care, 2003. 6(6): p. 635-8.
5. Halton, T. L. and F. B. Hu, The effects of high protein diets on thermogenesis, satiety and weight loss: a critical review. J Am Coll Nutr, 2004. 23(5): p. 373-85.
6. Beulens J W, Bindels J G, de Graaf C, Alles M S, Wouters-Wesseling W. Alpha-lactalbumin combined with a regular diet increases plasma Trp-LNAA ratio. Physiol Behav. 2004 June; 81(4):585-93.
7. Stunkard, A. J. and S. Messick. The three factor eating questionnaire to measure dietary restraint, disinhibition and hunger. J Psychosom Res. 1985. 29(1): p. 71-83.

1. Use of collagen hydrolysate for the preparation of an edible composition for limiting voluntary food intake, wherein the edible composition is essentially free from a natural tryptophan source.

2. Use according to claim 1, wherein the edible composition is for reducing a feeling of hunger.

3. Use according to claim 1, wherein the edible composition is for treatment and/or prevention of obesity.

4. Use according to claim 1, wherein the edible composition is administered once to three times a day.

5. Use according to claim 1, wherein the edible composition replaces one meal per day, preferably a breakfast.

6. Use according to claim 1, wherein the edible composition provides for 1-50% of the advised total calories intake per day.

7. Use according to claim 1, wherein the edible composition provides 5-200 grams of collagen hydrolysate per day.

8. An edible composition comprising collagen hydrolysate for use in a method for treatment and/or prevention of obesity, wherein the edible composition is essentially free from a natural tryptophan source.

9. An edible composition comprising:
a. collagen hydrolysate, in such an amount that it provides at least 2-50% of the total calories of the composition, and
b. optionally, another protein source, which is not a tryptophan source, and
c. a fat source, in such an amount that it provides between 0.1 and 50% of the total calories of the composition, and
d. a carbohydrate source, in such an amount that it provides between 0.1 and 85% of the total calories of the composition; the edible composition being essentially free from a natural tryptophan source.

10. The composition according to claim 9, said composition further being essentially free of L-tryptophan.

11. The composition according to claim 9, wherein the carbohydrate source further comprises a dietary fiber.

12. The composition according to claim 9, said composition being in the form of a unit dose and providing from 5 to 200 grams, preferably from 10 to 100 grams of collagen hydro lysate per said unit dose.

13. The composition according to claim 9, said composition being a solid or semi-solid food product.

14. The food product according to claim 13, said product being a nutritional bar.

15. The composition according to claim 13, said composition being a reconstitutable powder.

16. The composition according to claim 9, wherein the composition is a liquid or spoonable food product, such as a custard, a pudding, a soup, or a shake.

17. The composition according to claim 9, wherein the collagen hydrolysate has an average molecular weight of between 1 and 20, more preferably between 2 and 10 kDalton.
